(19) United States
(12) Patent Application Publication
(10) Pub. No.: US 2023/0077649 A1
(43) Pub. Date: Mar. 16, 2023

(54) Malodor Suppression in Brassica Amidoalkyl Containing Cosmetic Compositions

(71) Applicants: Lisa Argus, Miami, FL (US); Richard Rigg, Richmond Hill, NY (US)

(72) Inventors: Lisa Argus, Miami, FL (US); Richard Rigg, Richmond Hill, NY (US)

(21) Appl. No.: 17/888,587

(22) Filed: Aug. 16, 2022

Related U.S. Application Data
(60) Provisional application No. 63/260,882, filed on Sep. 3, 2021.

(51) Int. Cl. A61K 8/42; A61K 8/81; A61K 8/14

(52) U.S. Cl. CPC A61K 8/42; A61K 8/8135; A61K 8/14

(57) ABSTRACT

A cosmetic composition is provided including a brassica amidoalkyl compound which emits a malodor, a malodor suppressant which is Hydrogenated Castor Oil/Sebacic Acid Copolymer; and a cosmetically acceptable carrier.

This application claims benefit of U.S. Provisional Patent Application Ser. No. 63/260,882 filed Sep. 3, 2021, herein incorporated by reference. The invention concerns malodor suppression in brassica amidoalkyl containing cosmetic compositions.

### BACKGROUND OF THE INVENTION

### Field of the Invention and Related Applications

### The Related Art

Cosmetics compositions are often judged on their sensory properties. They need to feel smooth, light and non-sticky. Moreover they need to smell pleasant. In fact, smell is the very first sensory impacting a consumer.

Some types of compounds develop malodor from degradation during storage. We have noted that brassica amidoalkyl compounds and also compositions containing them have quite a malodorous scent.

### SUMMARY OF THE INVENTION

A cosmetic composition is provided which includes:

(i) a brassica amidoalkyl compound which emits a malodor;

(ii) a malodor suppressant which is Hydrogenated Castor Oil/Sebacic Acid Copolymer; and

(iii) a cosmetically acceptable carrier.

### DETAILED DESCRIPTION OF THE INVENTION

Now we have found that Hydrogenated Castor Oil/Sebacic Acid Copolymer is a useful malodor suppressant. The copolymer is particularly effective at neutralizing odors arising from brassica amidoalkyl compounds and their degradation materials.

Brassica (such as in brassica alcohols) is a mixed group of alkyl radicals, particularly C16 to C24. The relative amount of C18:C22 found in the brassica section may range in a weight ratio from about 100:1 to 1:100, preferably about 50:1 to 1:1, more preferably 1:2 to 2:1. The amidoalkyl section may have alkyls selected from the group consisting of methyl, ethyl, propyl, butyl, pentyl, hexyl, septyl, octyl, dodecyl and radical mixtures thereof. Amidopropyls are preferred. Further, the amidoalkyl section may be bonded to mono- and di-alkyl amine units. These alkyls of the amino units may be selected from the group consisting of methyl, ethyl, propyl and radical mixtures thereof. Most preferred is dimethylamine. Especially preferred is brassicamidopropyl dimethylamine.

Brassicamidopropyl dimethylamines are derived from Brassica Campestris (rapeseed) Oil and commercially available from Inolex Corporation under the trademark ProCondition 22™.

Concentrations of brassica amidoalkyls may range from about 0.005 to 3%, usefully from about 0.01 to 2%, and particularly from about 0.01 to 1% by weight of the overall cosmetic composition.

Hydrogenated Castor Oil/Sebacic Acid Copolymer can be sourced from Croda Europe Ltd under the trademark Crodabond™.

Concentrations of the Hydrogenated Castor Oil/Sebacic Acid Copolymer may range from about 0.01 to 3%, usefully from about 0.1 to 2%, and more usefully from about 0.8 to 1.2% by weight of the composition.

Amounts of brassica amidoalkyls relative to Hydrogenated Castor Oil/Sebacic Acid Copolymer may range in a weight ratio from about 1:1 to 1:50, advantageously from about 1:1 to 1:8; and especially from about 1:1 to 1:15.

Advantageously, liposomes may be formulated as carriers for brassica amidoalkyls. Illustrative are lecithin and other phospholipids, polyethyleneglycol fatty acyl glycerides, and polyglyceryl polyethoxylated mono- and diglycerides. Most preferred are the polyglyceryl-10 dilinoleate and polyglyceryl-10 dioleate emulsifiers. These are described in U.S. Pat. No. 10,702,475 B2 (Rigg et al). Liposomes based on the aforementioned technology are commercially available from NextStep Laboratories Inc. in New York.

Cosmetic compositions of this invention also include a cosmetically acceptable carrier. Amounts of the carrier may range from 1 to 99.9%, preferably from 70 to 95%, optimally from 80 to 90% by weight of the composition. Among the useful carriers are water, emollients, fatty acids, fatty alcohols, humectants, thickeners and combinations thereof. The carrier may be aqueous, anhydrous or an emulsion. Preferably the compositions are aqueous, especially water and oil emulsions of the W/O or O/W or triplex W/O/W variety. Water when present may be in amounts ranging from 5 to 95%, preferably 20 to 70%, optimally from 35 to 60% by weight of the cosmetic composition.

Water when present as carrier or otherwise may advantageously be incorporated into the compositions as a deionized, sterilized, or pasteurized liquid or can be heat treated or irradiated after having been mixed with other components of the composition. These treatments insure elimination of pathogenic microbes.

Emollient materials may serve as cosmetically acceptable carriers. These may be in the form of silicone oils, synthetic or natural esters, hydrocarbons, fatty acids and fatty alcohols. Amounts of the emollients may range anywhere from 0.1 to 95%, preferably between 1 and 50% by weight of the cosmetic composition.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicone atoms.

Nonvolatile silicone oils useful as an emollient material include polyalkyl siloxanes, polyalkylaryl siloxanes and polyether siloxane copolymers. The essentially nonvolatile polyalkyl siloxanes useful herein include, for example, polydimethyl siloxanes having viscosities from about 5×10⁻⁶ to 0.1 m²/s at 250° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:

1) Alkyl esters of saturated fatty acids having from 10 to 24 carbon atoms. Examples include behenyl neopentanoate, isononyl isonanonoate, isopropyl myristate and octyl stearate.

2) Ether-esters such as fatty acid esters of ethoxylated saturated fatty alcohols.

3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

4) Wax esters such as beeswax, spermaceti wax and tribehenin wax.

5) Sugar esters of fatty acids such as sucrose polybehenate, sucrose polycottonseedate, sophorolips and rhamnolipids.

Hydrocarbons which are suitable cosmetically acceptable carriers include petrolatum, mineral oil, C₁₁-C₁₃ isoparaffins, and especially isohexadecane, available commercially as Permethyl 101A from Presperse Inc.

Fatty acids having from 10 to 30 carbon atoms may also be suitable cosmetically acceptable carriers. Illustrative of this category are pelargonic, lauric, myristic, palmitic, stearic, isostearic, oleic, hydroxystearic and behenic acids.

Fatty alcohols having from 10 to 30 carbon atoms are another useful category of a cosmetically acceptable carrier. Illustrative of this category are stearyl alcohol, lauryl alcohol, myristyl alcohol and cetyl alcohol.

Humectants of the polyhydric alcohol-type can be employed as cosmetically acceptable carriers. Typical polyhydric alcohols include glycerol, polyalkylene glycols, and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of humectant may range anywhere from 0.5 to 50%, preferably between 1 and 15% by weight of the composition.

Thickeners can be utilized as part of the cosmetically acceptable carrier of compositions according to the present invention. Typical thickeners include Carbomers such as crosslinked acrylates (e.g. Carbopol 9820), hydrophobically-modified acrylates (e.g. Carbopol 13820); cellulosic derivatives; and natural gums. Among useful cellulosic derivatives are sodium carboxymethylcellose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin, alginate and combinations of these gums. Inorganics may also be utilized as thickeners, particularly clays such as bentonites, montmorillonites, illites and hectorites, fumed silicas and silicates such as magnesium aluminum silicate (Veegum®). Amounts of the thickener may range from 0.0001 to 10%, usually from 0.001 to 1%, optimally from 0.01 to 0.5% by weight of the cosmetic composition.

Sunscreens may be formulated into the compositions of this invention. They may be organic or inorganic. Included can be UVA and/or UVB spectrum protective ranges. Organic sunscreens will have at least one chromophoric group absorbing within the ultraviolet ranging from 290 to 400 nm. Chromophoric organic sunscreens may be divided into the following categories (with specific examples) including: p-Aminobenzoic acid, its salts and derivatives (ethyl, isobutyl, glyceryl esters, p-dimethylaminobenzoic acid), Anthranilates (o-aminobenzoates, methyl, menthyl, phenyl, benzyl, phenylethyl, linalyl, terpinyl, and cyclohexenyl esters); Salicylates (octyl, amyl, phenyl, benzyl, menthyl, glyceryl, and dipropyleneglycol esters); Cinnamic acid derivatives (menthyl and benzyl esters, alpha-phenyl cinnamonitrile; butyl cinnamoyl pyruvate; Dihydroxycinnamic acid derivatives (umbellipherone, methylumbellipherone, methylaceto-umbellipherone); Trihydroxycinnamic acid derivatives (esculetin, methylesculetin, daphnetin, and the glucosides, esculin and daphnin); Hydrocarbons (diphenylbutadiene, stilbene); Dibenzalacetone and benzalacetophenone; Naphtholsulfonates (sodium salts of 2-napthol-3,6-disulfonic and of 2-napthol-6,8-disulphonic acids); Dihydroxynaphthoic acid and its salts; o- and p-Hydroxybiphenylsulfonates; Coumarin derivatives (7-hydroxy, 7-methyl, 3-phenyl); Diazoles (2-acetyl-3-bromoindazole, phenyl benzoxazole, methyl naphthoxazole, various aryl benzothiazoles); Quinine salts (bisulfate, sulfate, chloride, oleate and tannate); Quinoline derivatives (8-hydroxyquinoline salts, 2-phenylquinoline); Hydroxy- or methoxy-substituted benzophenones; Uric and vilouric acids; Tannic acid and its derivatives (e.g. hexaethylether); (Butyl carbityl) (6-propylpiperonyl) ether; Hydroxyquinone; Benzophenones (Oxybenzone, Sulisobenzone, Dioxybenzone, Benzoresorcinol, 2,2′,4,4′-Tetrahydroxybenzophenone, 2,2′-Dihydroxy-4,4′-dimethoxybenzophenone, Octabenzone; 4-lsopropyldibenzoylmethane; Butylmethoxydibenzoylmethane; Etocrylene; and 4-isopropylbenzoylmethane.

Particularly important sunscreens are: 2-ethylhexyl p-methoxycinnamate (available as Parsol MCX®), 4,4′-t-butylmethoxydibenzoylmethane (known commonly as Avobenzone, available as Parsol 1789®), octylsalicylate (available as Dermablock OS®), tetraphthalylidene dicamphor sulfonic acid (available as Mexoryl SX®), benzophenone-3 (Oxybenzone) and mixtures thereof.

Inorganic sunscreens are usually microfine particles of titanium dioxide and of zinc dioxide. “Microfine” is defined herein as average particle size ranging from 5 to 200 nm, usually from 10 to 50 nm.

Amounts of sunscreen may range from 0.01 to 20%, usually from 0.5 to 15%, and often from 4 to 12% by weight of the cosmetic composition.

Surfactants suitable for use may be those which can form emulsions and/or association structures. Surfactants can be characterized as being of the anionic, nonionic, cationic or amphoteric type. The term “surfactants” are defined herein to include materials otherwise called “emulsifiers”.

Examples of surfactants which may be used in the compositions described herein include salts of C8-C22 alkyl chain compounds. Representative are sodium tallowate, sodium cocoate, sodium alkyl sulfate (e.g. sodium lauryl sulphate and sodium myristyl sulfate), sodium N-acyl sarcosinates (e.g. sodium N-lauroyl sarcosinate and sodium N-myristoyl sarcosinate), sodium dodecylbenzenesulfonate, sodium hydrogenated coconut fatty acid monoglyceride sulfate, sodium lauryl sulfoacetate and N-acyl glutamates (e.g. N-palmitoyl glutamate), N-methylacyltaurin sodium salt, N-methylacylalanine sodium salt, sodium alpha-olefin sulfonate and sodium dioctylsulfosuccinate; N-alkylaminoglycerols (e.g. N-lauryl-diamino-ethylglycerol and N-myristyldiaminoethyl glycerol), N-alkyl-N-carboxymethylammonium betaine and sodium 2-alkyl-1-hydroxyethylimidazoline betaine; polyoxyethylenealkyl ether, polyoxyethylene alkyl aryl ether, polyoxyethylene lanolin alcohol, polyoxyethylene glyceryl monoaliphatic acid ester, polyoxyethylene sorbitol aliphatic acid ester, polyoxyethylene aliphatic acid ester, higher aliphatic acid glycerol ester, sorbitan aliphatic acid ester, and polyoxyethylene sorbitan aliphatic acid esters such as polyoxyethylene sorbital monooleate and polyoxyethylene sorbitan monolaurate. Bio-based surfactants useful herein are the rhamnolipids, alkyl polyglucosides, and sophorolipids.

Oil continuous (W/O) emulsions are created by dimethicone copolyols. A suitable example of such an emulsifier is PEG-8 Dimethicone which arises from polyethylene glycol derivative of Dimethicone containing an average of 8 moles ethylene oxide. Another useful oil continuous phase emulsifier is Gransurf®2106 is Dimethicone (and) PEG-10 Dimethicone Crosspolymer available from Grant Industries.

The surfactants/emulsifiers can be used at levels from 0.001% to 20%, preferably from 0.1% to 15%, more preferably from 1% to 10% and most preferably from 2% to 8% by weight of the cosmetic composition.

Preservatives may be incorporated into the cosmetic compositions to protect against the growth of potentially harmful microorganisms. Suitable preservatives include benzyl alcohol, hydantoin derivatives, propionate salts, sorbate salts and a variety of quaternary ammonium compounds. Cosmetic chemists are familiar with appropriate preservatives and routinely choose them to satisfy the preservative challenge test and to provide product stability. Particularly preferred preservatives are methylchloroisothiazolinone and methylisothiazolinone combinations, parabens, imidazolidinyl urea, sodium dehydroacetate and benzyl alcohol. Preservatives may be employed in amounts ranging from 0.01% to 2% by weight of the cosmetic composition.

Desquamation agents may be present. Illustrative are the monocarboxylic acids. Monocarboxylic acids may be substituted or unsubstituted with a carbon chain length up to 16. Particularly preferred carboxylic acids are the alpha-hydroxycarboxylic acids, beta-hydroxycarboxylic or polyhydroxycarboxylic acids. The term “acid” is to include not only the free acid but also salts and the C1-C30 alkyl or aryl esters thereof and lactones generated from removal of water to form cyclic or linear lactone structures. Representative acids are glycolic, lactic, malic and tartaric acids. A representative salt that is particularly preferred is ammonium lactate. Salicylic acid is representative of the beta-hydroxycarboxylic acids. Amounts of these materials when present may range from 0.01 to 15% by weight of the cosmetic composition.

Preferred desquamation agents may be selected from the group consisting of glycolic acid, lactic acid, salicyclic acid, retinoic acid, retinol and mixtures thereof, and including salt forms thereof.

Antiperspirant skin care cosmetic compositions for use herein may include well known antiperspirant metal salts of aluminum, zinc, zirconium and zirconium aluminum mixtures of sulfates, chlorides, chlorohydroxides, tetrachlorohydrex glycinates, alums, formats, lactates, benzyl sulfonates, succinates and phenol sulfonates. Typical levels of antiperspirant metal salts range from 1% to 35%, preferably from 1.5% to 25% by weight of the composition.

Colorants may either be dyes or pigments. A distinction is usually made between a pigment, which is insoluble in its vehicle (resulting in a suspension), and a dye, which either is itself a liquid or is soluble in its vehicle (resulting in a solution). A colorant can act as either a pigment or a dye depending on the vehicle involved. In some cases, a pigment can be manufactured from a dye by precipitating a soluble dye with a metallic salt. The resulting pigment is called a lake pigment.

Among the more common dyes are Alizarin, Azophloxin, Chrysoidin, Congo Red, Fuchsin Acid, Gentian Violet, Janus Green, Methyl Red, Naphthol Green, Naphthol Yellow, Rose Bengal, Sudan II, Titan Yellow and combinations thereof. Amongst pigments, titanium dioxide and aluminum lakes (aluminum salts of dyes) are most common. Amounts of the colorant may, according to the type of cosmetic product (lipstick, foundation, hair treatment, etc) range from 0.000001 to 10%, usually from 0.01 to 5% by weight of the cosmetic composition.

Cosmetic compositions intended to be skin lighteners normally will be formulated with a skin lightening compound. Illustrative substances are placental extract, lactic acid, niacinamide, arbutin, kojic acid, hydroquinone, resorcinol and derivatives including 4-substituted resorcinols and combinations thereof. Amounts of these substances may range from 0.1 to 10%, preferably from 0.5 to 2% by weight of the composition.

Also included may be such materials as resveratrol, alpha-lipoic acid, ellagic acid, kenitin, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B, Ceramide 6 and Ceramide 7) as well as pseudoceramides are useful. Amounts of these materials may range from 0.000001 to 10%, preferably from 0.0001 to 1% by weight of the composition.

The cosmetic compositions may contain a peptide active selected from pentapeptides, derivatives of pentapeptides, and mixtures thereof. As used herein, “pentapeptides” refers to both the naturally occurring pentapeptides and synthesized pentapeptides. A pentapeptide derivative-containing composition is Matrxyl®, which is commercially available from Sederma, France. The pentapeptides and pentapeptide derivatives are preferably included in amounts from 0.000001% to 20% by weight of the composition.

Cosmetic compositions may include vitamins. Illustrative vitamins include Vitamin A (retinol), Vitamin B₂, Vitamin B₃ (niacinamide), Vitamin B₆, Vitamin B₁₂, Vitamin C, Vitamin D, Vitamin E (tocopherol), Vitamin K and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate, and tocopheryl linoleate. DL-panthenol and derivatives may also be employed. A particularly suitable Vitamin B₆ derivative is Pyridoxine Palmitate. Flavonoids may also be useful, particularly glucosyl hesperidin, rutin, and soy isoflavones (including genistein, daidzein, equol, and their glucosyl derivatives) and mixtures thereof. Total amounts of vitamins or flavonoids when present may range from 0.0001 to 10% by weight of the composition.

The cosmetic compositions may be formulated into a wide variety of product types that include but are not limited to solutions, suspensions, lotions, creams, gels, toners, sticks, sprays, ointments, cleansing liquid washes and solid bars, shampoos and hair conditioners, pastes, foams, powders, mousses, shaving creams, wipes, strips, patches (transdermal or non-transdermal), electrically powered patches, wound dressing and adhesive bandages, hydrogels, film-forming products, facial and skin masks, make-up such as foundations, eye liners and eye shadows.

Except in the examples, or where otherwise explicitly indicated, all numbers in this description indicating amounts or material or conditions of reaction, physical properties of materials and/or use are to be understood as modified by the word “about”. All amounts are by weight of the final cosmetic composition, unless otherwise specified.

It should be noted that in specifying any range of concentration or amount, any upper concentration can be associated with any lower concentration or amount.

For the avoidance of doubt, the word “comprising” is intended to mean “including” but not necessarily “consisting of” or “composed of”. In other words, the listed steps or options need not be exhaustive.

“Cosmetic Composition” as used herein, is meant to include a composition for topical application to skin or hair of mammals, especially humans. Such a composition may be generally classified as leave-on or rinse-off, and includes any product applied to a human body for improving appearance, cleansing, or general aesthetics.

### Example 1

This example illustrates the invention in a mask formulation, particularly with brassicamidopropyl dimethylamine delivered within a liposome component.

| Ingredient | Weight % of Mask |
|---|---|
| Brassica Alcohol | 5.000 |
| Glycerin | 3.000 |
| Stearyl Alcohol | 2.500 |
| Cetyl Alcohol | 2.500 |
| Fragrance | 1.200 |
| Hydrogenated Castor Oil/Sebacic Acid Copolymer | 1.000 |
| Benzyl Alcohol | 0.800 |
| Brassicamidopropyl Dimethylamine* | 0.750 |
| Calcium Gluconate | 0.750 |
| Tocopherol | 0.030 |
| Bisabolol | 0.030 |
| Polyglyceryl-10 Dioleate | 0.030 |
| Deionized Water | to 100 |

*Delivered within an aqueous liposome.

### Example 2

This example illustrates a hair treatment composition which includes a combination of brassicamidopropyl dimethylamine and Hydrogenated Castor Oil/Sebacic Acid Copolymer.

| | Weight % of |
|---|---|
| Ingredient | Hair Treatment |
| Propanediol | 2.000 |
| Fragrance | 0.800 |
| Brassica Alcohol | 0.700 |
| Benzyl Alcohol | 0.600 |
| Guar Hydroxypropyltrimonium Salt | 0.455 |
| Brassicamidopropyl dimethylamine * | 0.380 |
| Hydrogenated Castor Oil/Sebacic Acid Copolymer | 0.380 |
| Ethylhexyl Glycerin | 0.200 |
| Sodium Gluconate | 0.190 |
| Calcium Gluconate | 0.190 |
| Potassium Gluconate | 0.190 |
| Xanthan Gum | 0.010 |
| Polyglceryl-10 Dipalmitate | 0.001 |
| Deionized Water | to 100 |

* Delivered within an aqueous liposome.

### Example 3

This example illustrates the invention in context of a shampoo formulation that combines a malodorous brassicamidopropyl dimethylamine with Hydrogenated Castor Oil/Sebacic Acid Copolymer to suppress malodor.

| | Weight % of |
|---|---|
| Ingredient | Shampoo |
| Glycerin | 6.100 |
| Na Lauroyl Methyl Isethionate | 5.350 |
| Na Cocoyl Isethionate | 4.900 |
| Lauramidopropyl Betaine | 4.000 |
| Fragrance | 1.200 |
| Acrylates Copolymer | 1.000 |
| Sodium Benzoate | 0.650 |
| Hydrogenated Castor Oil/Sebacic Acid Copolymer | 0.400 |
| Brassicamidopropyl Dimethylamine | 0.400 |
| Cetearyl Alcohol | 0.350 |
| Behenyl Alcohol | 0.350 |
| Glycol Distearate | 0.330 |
| Deionized Water | to 100 |

### Example 4

This example illustrates oil beads includable in hair formulations. The beads target and heal split hair ends. Any malodor generated by the brassicamidopropyl dimethylamine is suppressed by addition of Hydrogenated Castor Oil/Sebacic Acid Copolymer. Besides the presence of these materials, the beads may include meadowfoam seed oil, Abyssinian oil, camellia seed oil, tomato seed oil, lemon peel oil, lime oil, bergamot fruit oil, baobab oil, buriti fruit oil, pequi oil, and sunflower seed oil.

### Example 5

This example illustrates a lip color composition formulated with brassicamidopropyl dimethylamine and Hydrogenated Castor Oil/Sebacic Acid Copolymer.

| Ingredients | Weight % Lip Color |
|---|---|
| Triheptanoin | 49.000 |
| Candelilla Wax | 12.000 |
| Mica | 10.000 |
| Brassica Glycerides | 8.000 |
| Brassicamidopropyl dimethylamine | 5.000 |
| Tristearin/tribehenin | 4.900 |
| Titanium Dioxide | 2.000 |
| Mica/Titanium Dioxide/Iron Oxide/Carmine | 2.000 |
| Caprylyl Glycol | 1.000 |
| Carmine | 0.800 |
| Tocopherol | 0.300 |
| Hydrogenated Castor Oil/Sebacic Copolymer | 0.300 |

### Example 6

This example illustrates a nail strengthening liquid incorporating brassicamidopropyl dimethylamine and its malodor suppressant Hydrogenated Castor Oil/Sebacic Acid Copolymer.

| | Weight % of the |
|---|---|
| Ingredient | Nail Strengthener |
| Brassicamidopropyl dimethylamine | 0.400 |
| Hydrogenated Castor Oil/Sebacic Acid Copolymer | 0.400 |
| Ethanol | 5.000 |
| Jojoba Seed Oil | 2.000 |
| Fragrance | 1.000 |
| Tocopherol | 0.500 |
| Niacinamide | 0.500 |
| Brassica Glycerides | 90.200 |

### Example 7

Three compositions (7a, 7b, 7c) were prepared to demonstrate effectiveness of Hydrogenated Castor Oil/Sebacic Acid Copolymer in reducing malodor emanating from Brassica amides, particularly from Brassicamidopropyl Dimethylamine. Table I lists the ingredients of test samples 7a-7c.

TABLE I

| | Sample (weight %) | | |
|---|---|---|---|
| Ingredients | 7a | 7b | 7c |

Part A

| | | | |
|---|---|---|---|
| Deionized Water | 83.99 | 83.99 | 83.99 |
| Citric Acid (Anhydrous) | 0.24 | 0.24 | 0.24 |
| Hydroxyethyl Cellulose, Disodium | 0.20 | 0.20 | 0.20 |
| Phosphate, Sodium Phosphate, | | | |
| Polysorbate 60 | | | |

Part B

| | | | |
|---|---|---|---|
| Quaternium 91, Cetrimonium Methosulfate, | 6.00 | 6.00 | 6.00 |
| Cetearyl Alcohol | | | |
| Cetyl Alcohol | 5.00 | 5.00 | 5.00 |
| Brassicamidopropyl Dimethylamine | 2.00 | none | 2.00 |
| Hydrogenated Castor Oil/Sebacic Acid | none | 2.00 | 2.00 |
| Copolymer | | | |

Part C

| | | | |
|---|---|---|---|
| Isoamyl Laurate | 1.00 | 1.00 | 1.00 |
| Benzyl Alcohol, Ethylhexylglycerin, | 1.00 | 1.00 | 1.00 |
| Tocopherol | | | |

Part D

| | | | |
|---|---|---|---|
| Citric Acid | 0.030 | 0.030 | 0.030 |
| Deionized Water | 0.54 | 0.54 | 0.54 |

Part A was charged to a main reactor (3000 mL), heated to 80-85° C. and mixed with a pitched blade at 200 rpm. The hydroxyethylcellulose, disodium and sodium phosphate, polysorbate component was sprinkled into the mixing water vortex in the main reactor. The Quaternium 91, Citramonium Methosulfate, Cetearyl Alcohol ingredient was added to the main reactor with mixing at 80-85° C. and held at this temperature range for 30 minutes. Next the mixture was allowed to cool to 70-75° C. whereupon remaining Part B ingredients were added with mixing. Part C was added when mixture had cooled to 40-45° C. Part D was dosed at 35-40° C. to adjust pH to about 5.1-5.2. The resultant viscous liquid was then allowed to cool to room temperature.

A panel of experts was convened. A majority of the panel agreed that the most offensive odor emanated from sample 7a, the one having brassicamidopropyl dimethylamine (BD). With addition of Hydrogenated Castor Oil/Sebacic Acid Copolymer (CO), as in sample 7c, the malodor was hard to detect. Sample 7b served as control and had the least malodor. Therefor, the conclusion was that CO is an effective malodor suppressant against brassica type chemicals.

It will be appreciated by those skilled in the art that changes could be made to the embodiments described above without departing from the broad inventive concept thereof. It is understood, therefore, that this invention is not limited to the particular embodiments disclosed. Rather it is intended to cover modifications within the spirit and scope of the present invention as defined by the appended claims.

What is claimed is:

1. A cosmetic composition comprising:
(i) a brassica amidoalkyl compound which emits a malodor;
(ii) a malodor suppressant which is Hydrogenated Castor Oil/Sebacic Acid Copolymer; and
(iii) a cosmetically acceptable carrier.

2. A cosmetic composition according to claim 1 further comprising a liposome, the brassica amidoalkyl compound being held within the liposome.

3. A cosmetic composition according to claim 1 wherein amounts of brassica amidoalkyl compound relative to Hydrogenated Castor Oil/Sebacic Acid Copolymer may range in a weight ratio from about 1:1 to 1:50.

4. A cosmetic composition according to claim 1 wherein the amidoalkyl section comprises alkyls selected from the group consisting of methyl, ethyl, propyl, butyl, pentyl, hexyl, septyl, octyl, dodecyl and radical mixtures thereof.

5. A cosmetic composition according to claim 1 wherein the amidoalkyl is bonded to a mono-alkyl or di-alkyl amine unit.

6. A cosmetic composition according to claim 5 wherein alkyls of the amine units are selected from the group consisting of methyl, ethyl, propyl and radical mixtures thereof.

7. A cosmetic composition according to claim 1 wherein the brassica amidoalkyl compound is brassicamidopropyl dimethylamine.

8. A cosmetic composition according to claim 1 wherein the brassica amidoalkyl compound is present in an amount ranging from about 0.005 to 3% by weight of the composition.

9. A cosmetic composition according to claim 1 wherein the Hydrogenated Castor Oil/Sebacic Acid Copolymer is present in an amount ranging from about 0.01 to 3% by weight of the composition.

10. A cosmetic composition according to claim 1 wherein malodor emitted by the brassica amidoalkyl is suppressed by the Hydrogenated Castor Oil/Sebacic Acid Copolymer.

11. A method of suppressing malodor emitted from a brassica amidoalkyl compound formulated into a cosmetic composition, the method comprising adding an effective amount to reduce malodor of Hydrogenated Castor Oil/Sebacic Acid Copolymer to the composition.
